(19) United States
(12) Patent Application Publication
(10) Pub. No.: US 2011/0097392 A1
(43) Pub. Date: Apr. 28, 2011

(54) ANTIBODY BOUND SYNTHETIC VESICLE CONTAINING MOLECULES FOR DELIVER TO CENTRAL AND PERIPHERAL NERVOUS SYSTEM CELLS

(75) Inventors: Kevin Ka-wang Wang, Gainesville, FL (US); Jonathan Wang, Galnesville, FL (US); Jared V. Goodman, Gainesville, FL (US); Stephen Frank Larner, Newberry, FL (US)

(73) Assignee: BANYAN BIOMARKERS, INC

(21) Appl. No.: 12/988,479

(22) PCT Filed: Apr. 17, 2009
(86) PCT No.: PCT/US09/40982

§ 371 Date: Jan. 5, 2011

Related U.S. Application Data
(60) Provisional application No. 61/045,748, filed on Apr. 17, 2008.

(51) Int. Cl. A61K 9/127; C12N 5/0793; C12N 9/99; A61K 47/42; A61K 9/00; A61K 39/395; A61P 25/00

(52) U.S. Cl. 424/450; 435/375; 435/184; 514/773; 424/400; 424/178.1

(57) ABSTRACT

A process is provided of delivering at least one active agent cargo molecule into an neuronal cell whereby a cargo molecule is placed within a synthetic vesicle such as a liposome and a biotinylated protein such as an antibody is bound to the synthetic vesicle to form a protein bound synthetic vesicle whereby the protein recognizes a receptor expressed on the surface of a neuronal cell, and exposing the protein bound synthetic vesicle to the cell until the cargo molecule is delivered into the neuronal cell. Numerous cargo molecules are delivered by the inventive synthetic vesicle including a calpain inhibitor and a caspase inhibitor. The protein illustratively targets a cellular receptor for a ligand such as glutamate, glycine, dopamine, nicotine, muscarine, acetylcholine, or serotonin, and the like.

### CROSS REFERENCE TO RELATED APPLICATIONS

This application claims priority to U.S. Provisional Application No. 61/045,748 filed Apr. 17, 2008; of which is incorporated herein by.

### FIELD OF THE INVENTION

The present invention relates generally to a synthetic vesicle targeted to cells of the central nervous system (CNS) or peripheral nervous system (PNS) and, in particular, an antibody bound synthetic vesicle for delivery of molecules contained within the vesicle to a cell expressing a receptor target of the antibody.

### BACKGROUND OF THE INVENTION

Every year about 270,000 people experience moderate to severe traumatic brain injury (TBI). TBI is “an acquired injury to the brain caused by an external physical force . . . resulting in impairments in one or more areas, such as cognition; language; memory; attention; reasoning; abstract thinking; judgment; problem-solving; sensory, perceptual, and motor abilities; psycho-social behavior; physical functions; information processing; and speech.” TBI can result from many injury types illustratively including transportation accidents, acts of violence, sports injuries, physiological anomaly, or illness; alcohol is involved in over half of physical injury occurrences.

TBI is the leading cause of death and disability in persons under 45 years of age in industrialized countries (McAllister, 1992). Of the 1.5 million head traumas estimated to occur each year in the United States, 500,000 are likely to require hospitalization, and 80,000 result in some form of chronic disability (Langlois et al., 2006). The Center for Disease Control (CDC) estimates that at least 5.3 million Americans, or about 2% of the population, currently have a long-term requirement for assistance with daily living activities as a result of TBI (Langlois et al., 2006). Furthermore, total health costs for TBI amount to roughly $35 billion annually (Max et al., 1991). Despite the prevalence and severity of this form of injury, no effective treatment has yet been developed.

TBI is associated with many long-term disabilities such as Alzheimer's and Parkinson's disease, dementia puglistica, and post-traumatic dementia. Not all of the damage from TBI occurs at the moment of injury, but through secondary damage. Secondary brain damage can be, for example, the result of metabolic processes such as the lack of oxygen to the brain after the initial injury. One of the major causes of secondary brain damage is the increased levels of glutamate throughout the brain. Glutamate binds to the glutamate receptors that can be found on brain cell surfaces, allowing calcium to enter the intracellular fluid, cytoplasm. When too much calcium enters a brain cell, it leads to a number of events that eventually kill the cell. These events include the increase in the number of reactive oxygen species, mitochondrial dysfunction, excessive protease and phospholipidase activation, and calcium induced calcium release. All of these events lead to necrosis and apoptosis (two forms of programmed cell death) (Wang and Yuen (1994). Trends Pharmacol. Sci. 15, 412-419). Similar CNS and PNS cell perturbation, injury or death can also occur in other human diseases such as stroke, subarachnoid hemorrhage, epilepsy, seizures, neuropathic pain, headaches, Parkinson's, Alzheimer's disease, Huntington disease, multiple sclerosis.

There are a number of drugs that can prevent or mitigate secondary damage events from occurring. One such family of drugs includes protease inhibitors. One problem with the protease inhibitors is that, even though they are known to be cell protective, they have poor pharmacokinetics and dynamics owing to physiological instability. In addition, a patient is at risk of numerous undesirable side effects if the body is exposed to excessive amounts of these agents. Finally, it is difficult to administer multiple drugs at the same time. By elucidating biochemical promoter or inhibition signals responsive to brain cells after physical or chemical stresses the efficacy of one or more of a battery of prospectively effective compounds can be elucidated. By way of examples, calcium-activated cytosolic protease calpain is known to be activated in pro-necrotic cell injury while caspases are known to be activated in pro-apoptotic cell injury and are implicated in degrading key structural proteins of brain cells, leading to tissue auto-digestion as part of a TBI cascade.

These problems have the potential of being solved with the use of targeted and/or protective delivery. For example, cancer researchers have been trying to develop targetable molecules for use in chemotherapy that can be specifically directed to cell type, or cell phenotype so that they discriminately kill cancer cells as this would increase the effectiveness of the cancer treatment while simultaneously decreasing the risk of adverse events or other side effects. However, TBI presents unique problems that are not properly addressed by prior studies. For example, delivery systems are required that can recognize neuronal tissue that is physiologically normal or has been subject to primary or secondary injury mediators. Further, delivery systems are needed that can locate target neuronal tissue while discriminating against other tissue types and can be easily administered to a patient in need. Finally, the neuronal medium is unique and presents discernable challenges relative to other tissues.

Thus, there exists a need for compositions and processes for delivering cargo molecules to CNS and PNS cells in a subject with cellular specificity and with protection of the cargo molecule during transport.

### SUMMARY OF THE INVENTION

Provided is a process of delivering at least one active agent cargo molecule into an neuronal cell whereby a cargo molecule is placed within a synthetic vesicle such as a liposome and a biotinylated protein such as an antibody is bound to the synthetic vesicle to form a protein bound synthetic vesicle whereby the protein recognizes a receptor expressed on the surface of a neuronal cell, and exposing the protein bound synthetic vesicle to the cell until the cargo molecule is delivered into the neuronal cell. Numerous cargo molecules are delivered by the inventive synthetic vesicle including a calpain inhibitor and a caspase inhibitor. The protein illustratively targets a cellular receptor for a ligand such as glutamate, glycine, dopamine, nicotine, muscarine, acetylcholine, or serotonin, and the like.

In the inventive process the cargo molecule is optionally loaded into said synthetic vesicle before binding a biotinylated protein thereto. Also, a synthetic vesicle is optionally bound to a biotinylated protein with avidin or streptavidin intermediate there between. It is appreciated that exposing the neuronal cell is in vitro, ex vivo, or in vivo and that the cell is optionally in the central nervous system or peripheral nervous system of the subject.

Also provided is a composition that is optionally a synthetic vesicle having a volume and an exterior surface with a cargo molecule within the volume of the synthetic vesicle, a biotinylated antibody bound to the exterior surface of the synthetic vesicle wherein the antibody recognizes a receptor expressed on the surface of a neuronal cell. The antibody is preferably directed to a cellular receptor for a ligand such as glutamate, glycine, dopamine, nicotine, muscarine, acetylcholine, or serotonin, and the like. Preferably, the ligand is glutamate. Preferably the cargo molecule is a calpain inhibitor, a caspase inhibitor, or combinations thereof.

The inventive composition preferably is a liposome, polycaprolactone (PCL), or poly (lactic-co-glycolic acid) (PLGA). Further the synthetic vesicle is preferably bound to the antibody with an avidin or streptavidin intermediate there between. Preferably the synthetic vesicle is biotinylated.

Also provided is a method of treating a disease, injury or condition of a CNS cell whereby the inventive composition is administered to a subject. Preferably, the condition is traumatic brain injury. The cargo molecule is preferably an apoptosis inhibitor such as calpain inhibitor, a caspase inhibitor, or combinations thereof. Delivery the inventive compound preferably delivers the apoptosis inhibitor to the CNS cell exposed to trauma.

Also provided is a method for producing an inventive compound including forming a synthetic vesicle and incorporating a biotinylated phosphatidylethanolamine into said outer surface exclusively. Preferably, the cargo molecule is present while forming the synthetic vesicle. Also it is preferred that avidin or streptavidin is bound to the antibody prior to the antibody binding the synthetic vesicle.

### BRIEF DESCRIPTION OF THE DRAWINGS

FIG. 1 is a schematic that illustrates an inventive process;

FIG. 2 are Western blots that illustrate antibody recognition of receptors expressed on neuronal cells;

FIG. 3 are micrographs that illustrate antibody binding to cellular surfaces;

FIG. 4 are micrographs that illustrate coupling of streptavidin to cargo molecule encapsulated liposomes;

FIGS. 5A-C are micrographs that illustrate immunoliposomes binding to neuronal cell surfaces and internalization;

FIG. 6 is an electrophoretic gel that illustrates suppression of SBDP formation in challenged neurons;

FIGS. 7A-B are a bar graph and an electrophoretic gel respectively that illustrate quantitation of intact αII-Spectrin in cells challenged and administered an inventive liposome;

FIGS. 8A-B are micrographs that illustrate binding and uptake of immunoliposomes in neuronal cells; and

FIG. 9 is a bar graph that illustrates reduction in neuronal cell death by administration of immunoliposomes to challenged neuronal cells.

### BRIEF DESCRIPTION OF THE PREFERRED EMBODIMENTS

The present invention has utility in the delivery of a synthetic vesicle' s internal cargo to a cell by targeting the vesicle to the cell using an antibody. A vesicle cargo using dyes as examples which facilities cell imaging while a cargo of a therapeutic provides a targeted delivery and treatment to a cell with a negative condition or disorder. With the vesicle being internalized by the cell, the vesicle cargo will perform specific functions such as to protect the cells from programmed cell death (inhibitors) or other injury pathologies (DNA, siRNA, therapeutics) or to promote cell growth and replacement (stem cells or drugs to promote stem cell growth and/or differentiation).

The term “antibody” refers to an immunoglobulin which specifically binds to and is thereby defined as complementary with a particular spatial and polar organization of a species expressed on a cell surface and specifically includes a cell surface receptor as the target species for the antibody. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof.

An antibody, biotin, avidin, streptavidin, lipid, cargo molecule, or other molecule useful as a component of the present invention is optionally labeled. A person of ordinary skill in the art recognizes numerous labels operable herein. Labels and labeling kits are commercially available optionally from Invitrogen Corp, Carlsbad, Calif. Labels illustratively include, fluorescent labels, biotin, peroxidase, radionucleotides, colloidal gold, magnetic particles, enzymes, or other label known in the art.

The term “synthetic vesicle” refers to a hollow structure formed having a diameter of from 50 to 5,000 nanometers capable of carrying a cargo therein. Synthetic vesicles operative herein illustratively include liposomes and those formed of poly(lactic co-glycholic acid) (PLGA). Liposomes are completely closed structures composed of lipid bilayer membranes containing an encapsulated aqueous volume. Optionally, monolayers and micelles are also within the scope of the present invention. Preferably, a synthetic vesicle is a bilayer liposome. Liposomes may contain many concentric lipid bilayers separated by aqueous phase (multilamellar vesicles or MLVs), or may be composed of a single membrane bilayer (unilamellar vesicles).

The liposomes used in the present invention can have a variety of compositions and internal contents, and can be in the form of multilamellar, unilamellar, or other types of liposomes, or more generally, lipid-containing particles, now known or later developed. For example, the lipid-containing particles can be in the form of steroidal liposomes, U.S. Pat. No. 599,691, alpha-tocopherol containing liposomes, U.S. Pat. No. 786,740, stable plurilamellar liposomes (SPLVs), U.S. Pat. No. 4,522,803, monophasic vesicles (MPVs), U.S. Pat. No. 4,588,578, or lipid matrix carriers (LMC), U.S. Pat. No. 4,610,868, the pertinent portions of which are incorporated herein by reference. Within the class of liposomes that may be used in the present invention is a preferred subclass of liposomes characterized in having solute distribution substantially equal to the solute distribution environment in which prepared. This subclass may be defined as stable plurilamellar vesicles (SPLV), monophasic vesicles (MPVs), and frozen and thawed multilamellar vesicles (FATMLVs) as described in “Solute Distributions and Trapping Efficiencies Observed in Freeze-Thawed Multilamellar Vesicles” Mayer et al. Biochimica et Biophysica Acta 817:1983-196 (1985). It is believed that the particular stability of the SPLV type liposomes arises from the low energy state attendant to solute equilibrium.

Alternatively, techniques used for producing large unilamellar liposomes (LUVs), such as, reverse-phase evaporation, infusion procedures, and detergent dilution, can be used to produce the liposomes. A review of these and other methods for producing liposomes can be found in the text Liposomes, Marc J. Ostro, ed., Marcel Dekker, Inc., New York, 1983, Chapter 1, the pertinent portions of which are incorporated herein by reference.

In a preferred embodiment a liposome is an asymmetric bilayer. Illustratively, liposome bilayers are formed producing a symmetric bilayer after which components such as biotinylated PE are subsequently added to the outer membrane layer forming an asymmetric bilayer surface. The advantage of this method is that preformed and optionally preloaded symmetric vesicles can be formed using conventional techniques optimizing the loading of a cargo molecule(s) into the interior volume of the vesicle while simultaneously reducing vesicle aggregation effects.

A synthetic vesicle is optionally composed of a biotin-phospholipid incorporated into the surface of a liposome, a biotin-polyacid into the surface of a PLGA vesicle or a biotin-polyacid into the surface of a PCL vesicle. Vesicles are optionally produced from numerous phospholipid moieties illustratively including phosphatidylcholine, phosphatidylethanolamine, phosphatidylserine, PEGylated phospholipids, sphingomyelin, modifications thereof, and other lipids known in the art either phosphorylated or not. Phospholipids are illustratively from a variety of sources illustratively including soy, egg, or other source. Synthetic vesicles are optionally formulated with one or more sterols in the lipid layer(s). Preferably a sterol is cholesterol, but other sterols such as ergosterol, lanosterol, β-sitosterol, stigmasterol, and the like. It is further appreciated that modifications of sterols are similarly operative.

Synthetic vesicles are optionally solid lipid nanoparticles (SLN) or nanostructured lipid carriers (NLC) are two main types of lipid nanoparticles.

Biotinylated antibody or antibody immunogenic fragment are created that recognize antigens to cell expressed proteins such as glutamate receptors, metabotropic glutamate receptors (mGluR), and receptors that recognize ligands illustratively including glutamate, glycine, dopamine, nicotine, muscarine, acetylcholine and serotonin. Preferably, receptors are found on neuronal cells such as neural and glial cells illustratively including astrocytes and oligodendrocytes. The antibody is then coupled to the synthetic vesicles through the introduction of streptavidin or avidin to simultaneously bind the biotinylated antibody and biotinylated synthetic vesicle. In a preferred embodiment a synthetic vesicle is already preloaded with one or more types of cargo molecules for cellular delivery prior to binding an antibody or other protein.

Alternatively, the synthetic vesicles are optionally prepared in such a way as to create a transmembrane potential across the lamellae in response to a concentration gradient. This concentration gradient may be created by either sodium/potassium potential or pH. The difference in internal versus external potential is a possible mechanism which drives the loading of the synthetic vesicles with ionizable cargo molecules. It is appreciated that delayed loading of preformed synthetic vesicles then occurs in response to the transmembrane potential. These synthetic vesicles accordingly may be dehydrated in the presence of one or more protecting sugars such as the disaccharides trehalose and sucrose, stored in their dehydrated condition, and subsequently rehydrated with retention of the ion gradient and associated ability to accumulate the ionic cargo molecules.

An “immuno-synthetic vesicle” of which an immunoliposome is a subset is illustratively produced by (i) allowing liposomes to encapsulate cargo molecules such as fluorescent dyes (Hoechst-33258, Dextran-Fluorescein, and Dextran-Rhodamine Green); (ii) conjugating the liposomes to streptavidin using biotinylated phospholipids incorporated onto the liposome surface, and then (iii) conjugating this construct to, at least, one biotinylated antibody per synthetic vesicle. It is appreciated that streptavidin is preferably conjugated or bound to a biotinylated protein or antibody prior to exposure of the biotin-streptavidin bound antibody to a biotinylated synthetic vesicle.

An antibody is preferably specific to one of two neuron-specific receptors (glutamate receptor subtype N-methyl-D-Asparate Receptor-1 (NMDA-R1) or the Glycine-receptor) by having them bind to streptavidin incorporated during step (ii). The immuno-liposome preparation would then be incubated with cultured rat cerebrocortical or cerebellar granule neurons or differentiated neural PC-12 cells where the immune-liposomes would be examined for their ability to bind to the neuronal surface receptors via the antibodies coupled to the liposomes and optionally deliver the cargo molecules to the intracellular space or cytoplasm of the target cell. The entire liposome may optionally be internalized such that the encapsulated fluorescent dyes or other cargo molecules are released inside the neurons.

Several different types of inventive liposomes are optionally produced with variations in the types of antibodies, liposomes structural components (lipids) and encapsulated molecules (cargo molecules). Preferably, two types of antibodies, NMDA receptor antibody and glycine receptor antibody are employed. Both receptors that could be found on the surface of neuronal cells such as CGN or in vivo human or animal neurons. Liposomes are optionally formed either in the presence or the absence of cargo molecule. Types of fluorescent dye illustratively include Hoechst 33258, Dextran Rhodamine Green, and Dextran FITC. Empty liposomes are illustratively caused to uptake the dyes Hoechst 33258 and Dextran Rhodamine Green and the self-made liposomes were formed in the presence of and encapsulated the dye Dextran FITC (Table 1).

TABLE 1
Types of Dye-Encapsulated Liposomes Made

| Neuron | | Liposome | | |
|---|---|---|---|---|
| Targeting | | targeting | | Encapsulated |
| Molecule | Linker | molecule | Liposome | Molecule |
| Biotinylated | Texas Red | Biotinylated | Empty | Hoechst 33258 |
| NMDA-R | Avidin | Phosphatidyl- | Liposomes | Dextran |
| Antibody | | ethenalomine | | Rhodamine |
| | | | | Green |
| | | | Self Made | Dextran FITC |
| | | | Liposomes | |
| Biotinylated | | | Empty | Hoechst 33258 |
| Glycine-R | | | Liposomes | Dextran |
| Antibody | | | | Rhodamine |
| | | | | Green |
| | | | Self Made | Dextran FITC |
| | | | Liposomes | |

Each type of antibody is optionally attached to a streptavidin molecule with a Texas-Red dye molecule, and then connected to a dye encapsulated liposome through a biotinylated phospholipid (phosphotidylethenalomine) on the liposome surface. It is appreciated that other dyes or labeling molecules are also operable herein. Similarly, other lipid molecules with biotin anchors are operable herein. While biotin is a preferred linker molecule, other anchoring mechanisms are similarly operable.

Alternative anchoring mechanisms for binding an antibody to an inventive vesicle are similarly operable. Illustratively, anchoring mechanisms include those described by Leserman et al. (Liposome Technology, III, 1984, CRC Press, Inc., Ca., p. 29-40; Nature, 288, p. 602-604, 1980) and Martin et al., (J. Biol. Chem., 257, p. 286-288, 1982) which described procedures whereby thiolated IgG or protein A is covalently attached to lipid vesicles, and thiolated antibodies and Fab′ fragments are attached to liposomes, respectively. These protocols and various modifications (Martin et al, Biochemistry, 20, p. 4229-4238, 1981; and Goundalkar et al., J. Pharm. Pharmacol. 36, p. 465-466, 1984) represent the most versatile approaches to coupling. Avidin-coupled and avidin and biotinyl-coupled phospholipid liposomes containing actinomycin D have successfully targeted tumor cells expressing ganglio-N-triosylceramide (Urdal et al., J. Biol. Chem., 255, p. 10509-10516, 1980). Huang et al. (Biochim. Biophys. Acta., 716, p. 140-150, 1982) demonstrate the binding of mouse monoclonal antibody to the major histocompatibility antigen H-2 (K), or goat antibody to the major glycoprotein of Molony Leukemia Virus, to palmitic acid. These fatty acid modified IgGs were incorporated into liposomes, and the binding of these liposomes to cells expressing the proper antigens characterized. Also encompassed by the present invention is the covalent coupling using N-[4-(p-Maleimidophenyl)butyryl]phosphatidylethanolamine (MPB-PE) and N-[3(2-Pyridyldithio)proprionyl]phosphatidylethanolamine (PDP-PE) and other methods described in U.S. Pat. No. 5,171,578.

Cargo active agent molecules illustratively include therapeutics, drugs, cosmetics, diagnostic reagents, bioactive compounds, and the like. Specific examples of cargo molecules illustratively include dyes, apoptosis and oncosis inhibitors, anti-inflammatories, antineoplastics, small interfering RNA (siRNA), DNA, enzymes, nutrients, antipsychotics, cell growth factors (e.g., brain derived nerve growth factor (BDNF) and nerve growth factor (NGF)) and combinations thereof. A therapeutic operable in the subject invention is illustratively any molecule, compound, family, extract, solution, drug, pro-drug, or other mechanism that is operable for changing, preferably improving, therapeutic outcome of a subject at risk for or victim of a neuronal injury such as TBI. A therapeutic is optionally a muscarinic cholinergic receptor modulator such as an agonist or antagonist. An agonist or antagonist may by direct or indirect. An indirect agonist or antagonist is optionally a molecule that breaks down or synthesizes acetylcholine or other muscarinic receptor related molecule illustratively, molecules currently used for the treatment of Alzheimer's disease. Cholinic mimetics or similar molecules are operable herein. An exemplary list of therapeutics operable herein include: dicyclomine, scoplamine, milameline, N-methyl-4-piperidinylbenzilate NMP, pilocarpine, pirenzepine, acetylcholine, methacholine, carbachol, bethanechol, muscarine, oxotremorine M, oxotremorine, thapsigargin, calcium channel blockers or agonists, nicotine, xanomeline, BuTAC, clozapine, olanzapine, cevimeline, aceclidine, arecoline, tolterodine, rociverine, IQNP, indole alkaloids, himbacine, cyclostellettamines, derivatives thereof, pro-drugs thereof, and combinations thereof. A therapeutic is optionally a molecule operable to alter the level of or activity of a calpain or caspase. Such molecules and their administration are known in the art.

Preferably cargo molecules include luminescent and fluorescent dyes and calpain and caspase inhibitors. Preferably, a caspase inhibitor is a caspase-3 inhibitor. Calpain and caspase inhibitors can be obtained from sources known in the art such as EMD Chemicals Inc., Gibbstown, N.J. Illustrative examples of caspase inhibitors include: Z-D-DCB, Z-VAD(OMe)-FMK, Ac-VAD-CHO, Boc-Asp(OMe)-CH₂F, Z-Val-Ala-Asp-CH₂F, Ac-Val-Asp-Val-Ala-Asp-CHO, Bl-9B12, Z-Asp(OCH₃)-Glu(OCH₃)-Val-Asp(OCH₃)-FMK, Ac-Asp-Glu-Val-Asp-CHO, modifications thereof, combinations thereof, or other inhibitors illustratively known in the art. Illustrative examples of a calpain inhibitor include: SJA6017, Z-Val-Phe-CHO, Z-Leu-Leu-Tyr-CH₂F, Z-Leu-Nva-CONH-CH₂-2-Pyridyl, Z-Leu-Abu-CONH(CH₂)₃-morpholine (Abu=a-aminobutyric acid), Mu-Val-HPh-CH2F (Mu=morpholinoureidyl; HPh=homophenylalanyl), modifications thereof, combinations thereof, or other inhibitors known in the art. Active agent inhibitors of calpain and caspase delivered according to the present invention include those detailed in US 2008/0311036; WO08/0809969; WO08/048121; US2007/105917; and U.S. Pat. No. 7,001,770 B1.

The entrapment of two or more cargo molecules simultaneously may be especially desirable where such compounds produce complementary or synergistic effects. However, complementary or synergistic effects are not required. The amounts of drugs administered in liposomes will generally be the same as with the free drug; however, the frequency of dosing may be reduced.

These preparations may be administered to a subject for treatment of disease or injury. A subject illustratively includes a guinea pig, a hamster, a dog, a cat, a horse, a cow, a pig, a sheep, a goat, a chicken, non-human primate, a human, a rat, and a mouse. Subjects who most benefit from the present invention are those suspected of having or at risk for developing abnormal neurological conditions, such as victims of brain injury caused by traumatic insults (e.g., gunshot wounds, automobile accidents, sports accidents, shaken baby syndrome), ischemic events (e.g., stroke, cerebral hemorrhage, cardiac arrest), neurodegenerative disorders (such as Alzheimer's, Huntington's, and Parkinson's diseases; prion-related diseases; other forms of dementia), epilepsy, substance abuse (e.g., from amphetamines, Ecstasy/MDMA, or ethanol), and peripheral nervous system pathologies such as diabetic neuropathy, chemotherapy-induced neuropathy and neuropathic pain. Because the present invention preferably relates to human subjects, a preferred subject for the methods of the invention is a human being.

As used herein an injury is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An event is illustratively, a physical trauma such as an impact (percussive) or a biological abnormality such as a stroke resulting from either blockade or leakage of a blood vessel. An event is optionally an infection by an infectious agent. A person of skill in the art recognizes numerous equivalent events that are encompassed by the terms injury or event.

An injury is optionally a physical event such as a percussive impact. An impact is the like of a percussive injury such as resulting to a blow to the head that either leaves the cranial structure intact or results in breach thereof. Experimentally, several impact methods are used illustratively including controlled cortical impact (CCI) at a 1.6 mm depression depth, equivalent to severe TBI in human. This method is described in detail by Cox, CD, et al., J Neurotrauma, 2008; 25(11):1355-65. It is appreciated that other experimental methods producing impact trauma are similarly operable.

TBI may also result from stroke. Ischemic stroke is optionally modeled by middle cerebral artery occlusion (MCAO) in rodents. UCHL1 protein levels, for example, are increased following mild MCAO which is further increased following severe MCAO challenge. Mild MCAO challenge may result in an increase of protein levels within two hours that is transient and returns to control levels within 24 hours. In contrast, severe MCAO challenge results in an increase in protein levels within two hours following injury and may be much more persistent demonstrating statistically significant levels out to 72 hours or more.

Alternatively, the coupled synthetic vesicle preparations may be used in diagnostic assays. As used herein the term “diagnosing” means recognizing the presence or absence of a neurological or other condition such as an injury or disease. Diagnosing is optionally referred to as the result of an assay wherein a particular ratio or level of a biomarker is detected or is absent. Optionally, diagnosing is the presence or absence of a biological marker detectable prior to, during or following administration of the inventive compound.

As used herein the term “administering” or “exposing” is delivery of a therapeutic or other cargo molecule to a subject. The therapeutic is administered by a route determined to be appropriate for a particular subject by one skilled in the art. For example, the therapeutic is administered orally, parenterally (for example, intravenously), by intramuscular injection, by intraperitoneal injection, intratumorally, by inhalation, or transdermally. The exact amount of therapeutic required will vary from subject to subject, depending on the age, weight and general condition of the subject, the severity of the neurological condition that is being treated, the particular therapeutic used, its mode of administration, and the like. An appropriate amount may be determined by one of ordinary skill in the art using only routine experimentation given the teachings herein or by knowledge in the art without undue experimentation.

The mode of administration may determine the sites in a subject and cells to which the active agent molecules will be delivered. For instance, delivery to a specific site of infection may be most easily accomplished by topical application (if the infection is external e.g., on areas such as eyes, skin, in ears, or on afflictions such as wounds or burns) or by absorption through epithelial or mucocutaneous linings (e.g., nasal, oral, vaginal, rectal, gastrointestinal, mucosa, etc.). Such topical application may be in the form of creams or ointments. The inventive composition can be administered alone but will generally be administered in admixture with a pharmaceutical carrier selected with regard to the intended route of administration and standard pharmaceutical practice. They may be injected parenterally, for example, intravenously, intramuscularly, or subcutaneously. For parenteral administration, they are best used in the form of a sterile aqueous solution which may contain other solutes, for example, enough salts or glucose to make the solution isotonic.

For the oral mode of administration, liposome composition of this invention can be used in the form of tablets, capsules, lozenges, troches, powders, syrups, elixirs, aqueous solutions and suspensions, and the like. In the case of tablets, carriers which can be used include lactose, sodium citrate, and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, certain sweetening and/or flavoring agents can be added.

The delivery route of intrathecal may be particularly preferred to deliver cargo molecules to cells of the central nervous system-cerebrum in recognition of the impediment of transiting the blood-brain barrier. Delivery to peripheral nervous system cells by an intravenous route may be preferred. An inventive composition is administered orally, topically, parenterally, by inhalation or spray, sublingually, transdermally, via buccal administration, rectally, as an ophthalmic solution, or by other means.

Conventional adjuvants and pharmaceutical compounding agents for liposomal delivery are used to form a medicament suitable for administration to a subject. The subject is a human primate, non-human primate, rodent, dog, rabbit, or other domesticated mammal.

An inventive process is also provided for diagnosing and treating a multiple-organ injury. Multiple organs illustratively include subsets of neurological tissue such as brain, spinal cord and the like, or specific regions of the brain such as cortex, hippocampus and the like. Multiple injuries illustratively include apoptotic cell death which is detectable by the presence of caspase induced SBDPs, and oncotic cell death which is detectable by the presence of calpain induced SBDPs.

Treatment of a multiple organ injury in the inventive process is illustratively achieved by administering to a subject at least one therapeutic antagonist or agonist effective to modulate the activity of a protein whose activity is altered in response to the first organ injury, and administering at least one therapeutic agonist or antagonist effective to modulate the activity of a protein whose activity is altered in response to a second organ injury.

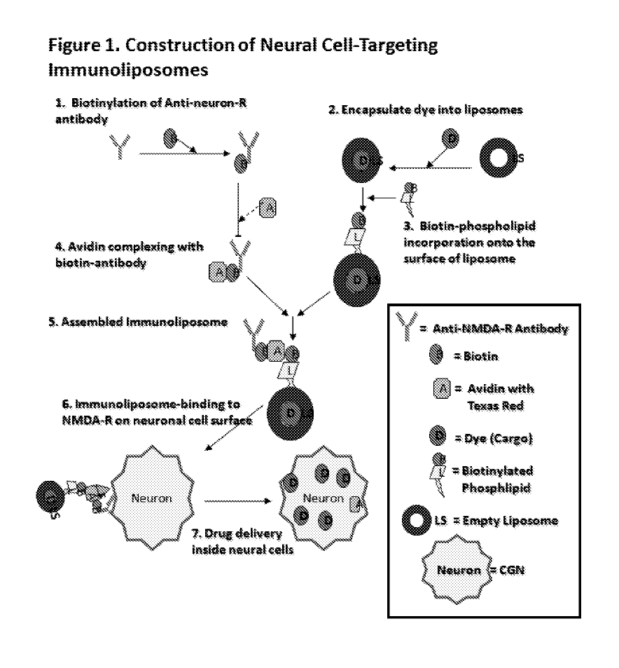

FIG. 1 presents an exemplary process for using immuno-liposomes as nanocarriers for the targeted delivery of protectants (such as neuroprotective drugs) to neurons or other CNS or PNS cells (such as glia cells, microglia cells or oligodendrocytes) in vivo, in vitro or ex vivo. FIG. 1 illustrates (i) coupling of liposome to antibody creating a shuttling device that targets neurons, by using a biotin-avidin-biotin bond, (ii) Biotin attachment to both antibody and liposome; (iii) antibody and liposome attachment to each other through molecule of streptavidin; (iv) Dye or other cargo molecule encapsulated into the liposome, and (v) Antibody binding to a cell surface receptor promoting liposome internalization in time releasing contents into cytoplasm.

Other potential neuro-receptors relevant to different neurological diseases as targets for antibody bound synthetic vesicle containing molecules for delivery to the CNS or PNS illustratively include: Glutamate/NMDA (type: ionotropic); Glutamate/Kainate (type: ionotropic); Glutamate/AMPA (non-NMDA) (type: ionotropic); mGluR (L-AP4, ACPD, L-QA) (type: metabotropic); Glycine; Dopamine; Nicotinic acetylcholine; and Serotonin/5-HT₃.

Illustrative examples of the numerous disorders such as diseases or injuries treatable by the subject invention include: TBI; stroke; spinal cord injury; subarachnoid hemorrhage; Parkinson's disease; attention-deficit/hyperactivity disorder; schizophrenia; drug/alcohol dependence; Myasthenia gravis; Alzheimer's disease; attention deficit disorder; depression; schizophrenia; sudden infant death syndrome; and migraines.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian cells, tissue, or subjects, a person having ordinary skill in the art recognizes that similar techniques and other techniques know in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained.

### EXAMPLE

### Example 1

Reagents and Antibody Target Recognition: Exemplary materials and reagents used are optionally as follows. It is appreciated that other reagents are similarly operable to make and use the present invention as recognized by persons of ordinary skill in the art. Locations for obtaining such reagents are similarly known to those of skill in the art such as from biological reagent suppliers including Invitrogen Corp. (Carlsbad, Calif.), EMD Chemical, Inc., VWR Scientific (West Chester, Pa.), Santa Cruz Biotechnology (Santa Cruz, Calif.), and the like. Materials and reagents illustratively include: PBS Solution, 3.7% formalin solution, Tris-glycine electrophoresis buffer, (Invitrogen), Gel transfer buffer (Invitrogen), precast electrophoresis gels (Invitrogen), Western blot filter paper and Polyvinylidene Difluoride (PVDF) membranes (Invitrogen), methanol, Tris-buffered saline with Tween-20 (TBST) solution, (Sigma), rat primary cerebellar granular neurons, non-fat dry milk, NMDA-receptor1 (NMDA-R1) (extracellular loop) primary antibody (Chemicon, #MAB363), Glycine receptor (Glycine-R) primary antibody (Gene Tex Inc.; #GTX30177), anti-rabbit IgG-biotinylated species-specific donkey secondary antibody (Amersham), anti-mouse IgG-biotinylated species-specific sheep secondary antibody (Amersham), streptavidin alkaline phosphatase conjugate tertiary antibody (Amersham), NBT and BCIP phosphatase substrate (KPL), SURELINK Chromophoric Biotin Labeling Kit (Pierce), cell culture media, DMEM solution (Sigma), phosphatidylcholine (Avanti Polar Lipids), biotinylated phosphatidylethanolamine, (Avanti Polar Lipids), cholesterol (Avanti Polar Lipids), chloroform, Dextran-Rhodamine Green, Dextrane-FITC, (Sigma; #46945), Hoechst 33258 dye (Sigma), HEPES buffer, liquid nitrogen, liposome extrusion kit, COATSOME Empty Liposomes (neutral) (NOF America corp.), Texas Red conjugated streptavidin (Rockland Inc.; #a003-09), and dialysis kit with membrane (M.W. 20,000 cutoff) (Millipore).

Antibody Binding to Receptor Target.

The reactivity of the NMDA-R (receptor) and glycine-R antibodies are tested against the NMDA-R and Glycienc-R present in rat cerebellar granule neuron (CGN) lysates. Proteins are extracted from lysed CGN optionally treated with an excessive amount of NMDA, a reagent toxic to the cells by causing cellular proteolysis and cell death such as oncosis (necrosis) or apoptosis. Lysates from control CGN cells are subjected to SDS-polyacrylamaide gel electrophoresis followed by immunoblotting on PVDF membranes. The membranes are probed with anti-NMDA-R, anti-Glycine-R and anti-αII-spectrin and β-actin (as control) antibodies. As seen in FIG. 2 the antibodies targeted the proper receptor proteins located on the cell membrane. The same technique is also used to study proteolytic breakdown products (SBDP) of a neuronal cytoskeletal protein alphaII-spectrin. Specific SBDPs have been associated with neuronal cell death in the form of necrosis, apoptosis and autophagic cell death (Wang (2000) Trends Neurosci. 23, 20-26; Sadasivan, S., Waghray, A., Lamer, S. F., et al. (2006) Apoptosis 11(9):1573-1582) (FIG. 2). Anti-NMDA-R antibodies detects the NMDA-R showing a protein band of 120 kDa while anti-Glycine-R antibody detects the Glycine-R showing a protein band of 60 kDa in the CGN lysate, respectively (FIG. 2). These molecular weights match those reported in the published literature for the respective receptors. In addition, αII-spectrin and β-actin acting as controls detect their respective proteins of 280 kDa and 43 kDa (FIG. 2). This experiment demonstrates that both NMDA-R and Glycine-R antibodies can specifically target their respective receptors in CGN cells.

### Example 2

Antibody recognition of NMDA/Glycine receptors on the surface of CGN cells by immunocytochemistry.

Antibody targeting the surface expressed NMDA-receptor or glycine-receptor is confirmed by immunocytochemistry using CGN cells. The CGNs are grown on glass cover slips for seven days. They are then washed with phosphate buffered saline (PBS) and fixed with a 4% paraformaldehyde solution for 10 minutes at 4° C. and then blocked with a 5% normal goat serum in TBST for 30 minutes at room temperature to prevent any non-specific binding of the antibody.

The cells are divided into two experimental groups. One group is washed with cold methanol for about one minute to break open the cell membrane and make the cells permeable to antibodies. In the other group, the cell membranes are left intact so that antibodies cannot cross the cell membrane. Both groups are incubated with the primary antibodies NMDA and glycine receptor antibodies (1/500) overnight and then washed and incubated with FITC-secondary antibody (1/1,000) for 1 hour in the dark. The nuclei of the cells are stained with a DAPI solution and the cells were observed under the microscope.

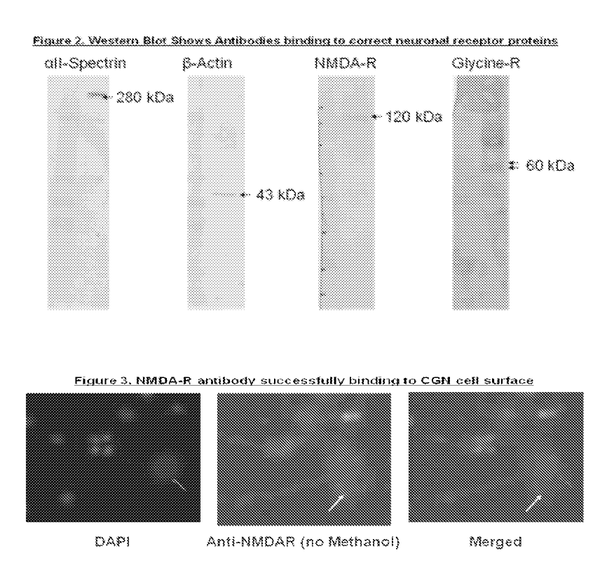

The NMDA-R antibody binds to NMDA-R of cell surface of non-permeabilized cell surface of cerebellar granule neurons (CGN) (yellow arrow). Glycine-R antibody also functions similarly (results not shown). DAPI shows cell nuclei (red arrow) (FIG. 3). The successful binding of the antibodies to the cells without permeablized membranes (not treated with methanol) indicates that the antibody is able to attach to a site on the receptor located on the cell surface.

### Example 3

### Coupling of Biotin to Antibodies

Inventive antibodies are biotinylated by methods known in the art. Briefly, the antibodies are transferred into a 1× Modification Buffer (100 mM phosphate, 150 mM NaCl, pH 7.2-7.4). A biotin solution is prepared at a concentration of 0.5 mg of biotin per 25 μL DMF (Dimethylformamide). 0.8 μL of biotin solution is added to the antibody solutions and incubated at room temperature for two hours on a rotational agitator. After incubation, the solutions are transferred to spin filters and centrifuged for 30 minutes at 12,000×g four times to filter out unbound biotin molecules. The remaining solution that had not passed through the filter is stored at 4° C. until further use.

### Example 4

### Construction of Dye-Encapsulated Liposomes

Liposomes are produced using a solution created by adding 50 mg of phosphatidylcholine and 0.0128 mg of cholesterol to 2 mL of chloroform in a boiling flask. The solution is stirred until all components are fully dissolved. The boiling flask is rotated sideways half submerged in a 50°-60° C. water bath until the chloroform is evaporated and a thin film of phosphatidylcholine and cholesterol forms on the bottom of the boiling flask. The flask is placed into a vacuum overnight to remove residual traces of chloroform. The formation of the film ensures that the phosphatidylcholine and the cholesterol are evenly distributed and will not clump up when they were redissolved into solution. A dye solution is then created by combining the dye (Dextran-FITC) with a HEPES buffer (4-(2-hydroxyethyl)-1-piperazineethanesulfonic acid) and combined with the phospholipid. The boiling flask is rotated at an angle half submerged until the phospholipid film completely dissolves into the dye solution. The solution is then frozen by insertion in liquid nitrogen for 2 minutes, thawed in a warm bath for 5 minutes, vortexed for 30 seconds, and then the entire process was repeated four times in order to break up any clumps of phospholipids that had formed. The liposomes encapsulating the dye solution are formed by extruding the phospholipid-dye solution through a series of membranes with decreasing pore sizes (400 nm, 200 nm, 100 nm) 10 times for each membrane. The extrusion through the membranes forces the phospholipids to form a phospholipid bilayer in the shape of a sphere, encapsulating the dye molecules in the hollow center of the sphere at the same time. The dye-encapsulated liposomes are then biotinylated by combining them with biotinylated phosphatidyl ethanolamine (1%) in a boiling flask. This process produces liposomes with biotin molecules on the outside of the phospholipid bilayer, which are conjugated to the biotinylated antibodies.

### Example 5

Encapsulating dye into pre-made empty liposomes.

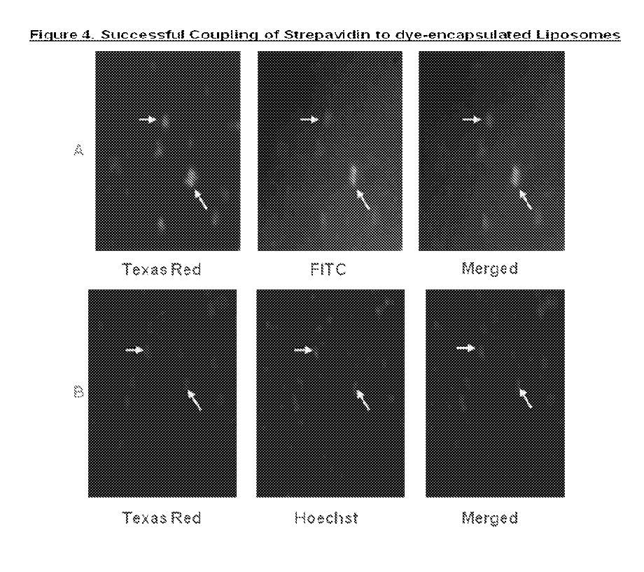

Another set of liposomes is produced from pre-made empty liposomes purchased in a dehydrated state. These liposomes are restored by adding the dye solution of Example 4, or distilled water and dye (Dextran-Rhodamine Green and Hoechst-33258) to the empty liposomes and then shaking gently to distribute the solution throughout the liposomes. These liposomes are also biotinylated through the same process as described in Example 4 with a fluorescent avidin molecule. Both sets of liposomes are tested for the proper encapsulation of dye using a high speed centrifuge to separate the liposomes from leftover dye molecules, and then using a spectrophotometer to determine the amount of dye in the liposomes and the successful association with streptavidin. Dye is successfully incorporated into the liposome. (See e.g. FIG. 4.)

### Example 6

### Conjugation of Liposomes to Antibodies

Liposomes are conjugated to the antibodies of Examples 1 and 2 (NMDA receptor and Glycine receptor antibodies) to create an immuno-liposome that is able to target the NMDA and Glycine receptors on neurons in vitro, ex vivo, or in vivo. Conjugation is effected by forming a bridge of streptavidin between biotinylated liposome and biotinylated antibody. Streptavidin has four binding sites for biotin molecules. Mixing the biotinylated liposomes, biotinylated antibodies, and streptavidin together in the proper ratio creates an inventive compound. Formation of immunoliposomes is achieved by combining a mixture of streptavidin with antibody. The solution is placed on an agitator for five minutes to conjugate the biotin on the antibodies to the streptavidin (the streptavidin optionally has a molecule of Texas Red dye attached to confirm conjugation by fluorescence measurements). Then the biotinylated dye-encapsulated liposomes of Examples 4 or 5 are added to the mixture, which is then placed on an agitator for five minutes to conjugate the biotin on the liposomes to the streptavidin binding sites that had not been occupied by the biotin on the antibodies. The final molecule is optionally termed a dye-encapsulated immunoliposome. The excess unbound avidin and liposomes are filtered out in a dialysis membrane submerged in PBS for 4 hours, replacing the PBS every hour. The remaining solution is tested for the presence of dye and streptavidin by using a spectrophotometer to examine the fluorescence of the dye and then the fluorescence of the streptavidin. (FIG. 4.)

Table 2A demonstrates dye molecule fluorescence in each set of immunoliposomes and Table 2B demonstrates the fluorescence emitted by the Texas Red molecule associated with the streptavidin on each set of immunoliposomes. Both tables indicate significant amounts of fluorescence in all sets of liposomes for both the dye in the liposomes and the Texas Red dye on the streptavidin. This fluorescence indicated the successful incorporation of dye and binding of streptavidin to the liposome because all free floating dye and streptavidin were removed when filtered through the dialysis membrane.

TABLE 2
Fluorescent readings showing successful packaging of all
components of dye-loaded avidin Antibody linked immunoliposomes

| (A) | | Rhodamine | FITC | Hoechst |
|---|---|---|---|---|
| | | Ex 502, | Ex 494, | Ex 359, |
| | | Em 527 | Em518 | Em 491 |
| | NMDA-R | 6905.7 | 65279.0 | 1512.5 |
| | antibody-conjugated | | | |
| | Liposomes | | | |
| | Glycine-R | 5864.7 | 64568.7 | 33514.7 |
| | antibody-conjugated | | | |
| | Liposomes | | | |
| (B) | | Dextran- | | |
| | Texas Red | Rhodamine G- | Dextran-FITC | Hoechst- |
| | Ex 596, Em 625 | encapsulated | encapsulated | encapsulated |
| | NMDA-R | 451.4 | 1038.0 | 530.5 |
| | antibody-conjugated | | | |
| | Liposomes | | | |
| | Glycine-R | 2739.1 | 2545.1 | 2850.2 |
| | antibody-conjugated | | | |
| | Liposomes | | | |

Fluorescent microscopy of the immunoliposomes shows that the Texas Red-linked streptavidin is in the same location as the Dextran-FITC-encapsulated liposomes with glycine-R antibody (FIG. 4A) and Hoechst-33258-encapsulated liposomes (with NMDA-R antibody) (FIG. 4B). Other immunoliposomes have similar results. When the two images are merged, the fluorescence of the dyes overlaps giving a yellow or orange color. Thus the streptavidin and liposomes are successfully conjugated.

This procedure unexpectedly and surprisingly avoids aggregation or precipitation of the liposomes prior to coupling with an antibody and allows for higher ratios of antibody/liposome that was previously achievable. (See e.g. U.S. Pat. No. 5,171,578.)

### Example 7

Specific association of immunoliposomes with cerebellar granule neurons (CGN) cells.

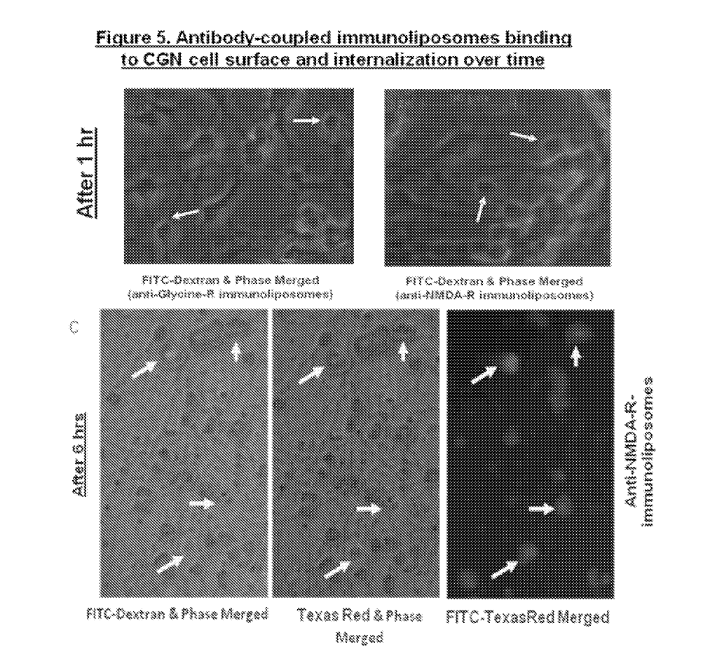

Immunoliposome specific association with neuronal cells is achieved by coupling the inventive immunoliposomes to CGN cell receptors. Immunoliposomes are added to a culture of CGN cells in 12-well plates under a sterilized fume hood to avoid contamination. The CGNs are then incubated at 37° C. in 5% CO₂ for 1 hour. At the 1, 4, and 24 hour time intervals, the cells are removed from the incubator and the liposome media is removed and saved. The remaining cells are washed twice with immunoliposome free media. After the first hour the cells are then observed and photographed under a microscope. The liposome media is replaced after microscopy observation and the cells were incubated again. The procedure is then repeated at the four hour and twenty-four hour time intervals. (FIG. 5A, B) After 1 hour, liposomes attach to their respective receptors on the surface of the cell. Evidence of this is in the fluorescent outline of the cell. The merged fluorescent images are of the FITC-Dextran and the phase contrast image of the CGN. The fluorescence comes from the FITC dye molecules inside the liposomes that are attached to the cell surface through the antibody binding to the receptor on the cell membrane. These are not free floating liposomes, as the media had already been removed and the cells washed with new media.

These results demonstrate that the liposomes are able to successfully attach to the cell surface through the use of the targeting antibodies. (FIG. 5C) After 6 hours, liposomes are internalized by the CGN cells (yellow arrow) as shown with both Dextran-green and Avidin-Texas Red fluorescence images which correspond with the locations of the cells. The contents of the liposome are released when the liposome phospholipid bilayer fuses with the membrane of the cell in a process similar to endocytosis. These results suggest that not only do the liposomes bind to the cell surface but that they are also able to release their dye cargo contents into the cell.

### Example 8

Delivery of calpain and caspase inhibitor cargo molecules decreases NDMA induced αII-Spectrin breakdown.

The calpain inhibitor (SJA6017) and caspase inhibitor (Z-D-DCB) (Wang 2000) are encapsulated into neuroreceptor-targeting immunoliposomes in place of the dye as in Examples 4 or 5. These liposomes are used to form immunoliposomes as in Example 6 and associated with CGN cells as in Example 7 where the cells were incubated in the presence or absence of excitotoxin, glutamate analogue, NMDA or neurotoxin (staurosporin). The cells are lysed and the presence of spectrin breakdown products (SBDP) is probed by western blotting.

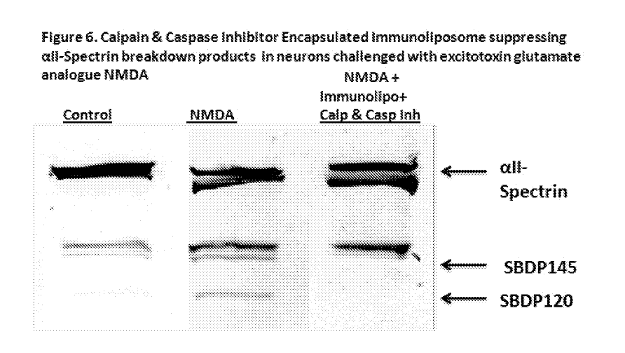

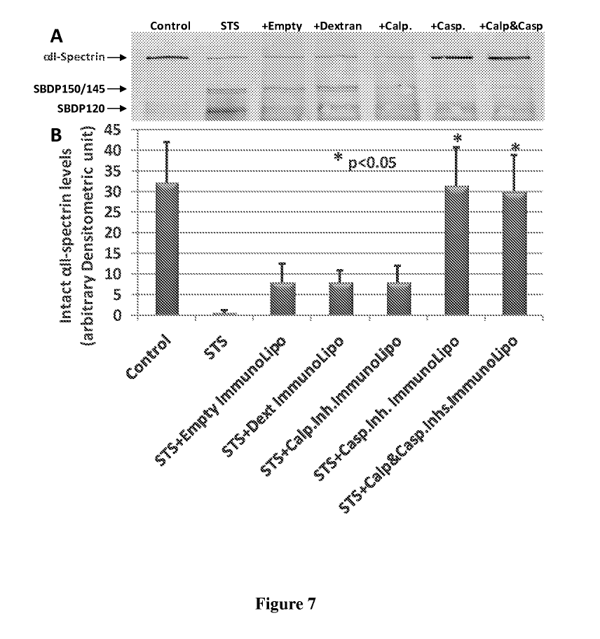

As observed in FIG. 6, when prototypic neuroprotective drugs such as calpain inhibitor (SJA6017) and caspase inhibitor (Z-D-DCB) are encapsulated into neuroreceptor-targeting immunoliposomes and delivered into neurons the presence of SBDP are reduced relative to control. In healthy rat PC-12 cells, αII-Spectrin exists predominantly on an immunoblot as an intense intact αII-Spectrin band and little spectrin breakdown product bands (SBDP 150/145 and SBDP120). Cells treated with STS (Staurosporin) alone for 12 h exhibit almost no intact αII-spectrin, but intense SBDP150/145 and SBDP120 bands (FIG. 6). When densitometric analysis is performed STS-treated cells show dramatically a dramatically decrease in the intact αII-spectrin levels when compared to control cells. (FIG. 7) Both caspase inhibitor and calpain+caspase inhibitors-loaded immunoliposomes provide a significantly enhanced protection for αII-spectrin from degradation, induced by neurotoxin apoptosis-inducing staurosporin (STS: 0.5 μM, for 24 h), in differentiated PC-12 cells. Statistical significance: p<0.05.

Empty immunoliposomes as well as Dextran-immunoliposomes serving as negative controls also exhibited a minimal amount of intact αII-spectrin, indicating a significant level of cell death. In contrast, the intact αII-spectrin protein is significantly preserved in the presence of immunoliposomes loaded with the caspase Inhibitor and the combined calpain and caspase Inhibitor (p<0.05, Student T-test), indicating the ability of the drug-loaded immunoliposomes in preventing cytoskeletal breakdown as a measure for the preventation of cell death. These data demonstrate that neuroprotection or the prevention of continuation of neuronal damage is achieved by the specific delivery of calpain and caspase inhibitors to neurons.

### Example 9

Calpain and caspase inhibitors are successfully delivered by immunoliposomes and taken up by cells.

PC-12 cells, except for the control and staurosporin (STS) alone conditions, are exposed to immunoliposomes for 4 hours and then exposed to the neurotoxin STS challenge for 12 hours. Staurosporine is used as the neurotoxic challenge as its neurotoxicity was previously established to mediate through the NMDA receptor pathway (Jantas-Skotniczna et al., 2006; Jantas et al., 2008) and that it activates both calpain and caspase cell death pathways (Nath et al., 1996; Wang et al., 1998).

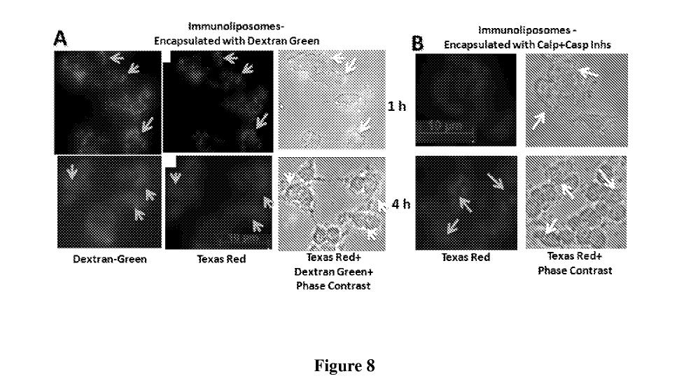

FIG. 8 demonstrates that drug/dye loaded immunoliposomes bind and internalize into differentiated PC-12 Cells monitoring Dextran Green labeled dye and Texas Red Avidin.

FIG. 8A demonstrates that Dextran-Green loaded immunoliposomes readily attach to PC-12 cell surfaces by 1 hr and are internalized into cell cytoplasm by 4 hr. FIG. 8B demonstrates that calpain and caspase inhibitors-loaded immunoliposomes behave similarly (detected via Texas Red Avidin).

### Example 10

Immunoliposome delivery of calpain and caspase inhibitors protect cells from damage.

PC-12 and CGN cells are exposed to immunoliposomes loaded with calpain+caspase inhibitors as in Examples 8 and 9. Cell death is quantified using a lactate dehydrogenase (LDH) assay. Cell media in the amount of 100 μL is collected from each well, transferred to a centrifuge tube and centrifuged at 14,000×g for 3 minutes. 50 μL of the supernatant from each tube is transferred to a well on a 96-well plate along with 50 μL of LDH assay solution (Promega) and the plate is left in the dark allowing the reaction to proceed for 30 minutes. Absorption at wavelength 490 nm was measured in a spectrophotometer (Molecular Device Spectramax 190) to quantify cell death. Release of cytosolic enzyme LDH into extracellular fluid (ECF) (i.e. cell media) was proportional to the amount of cell death (Koh and Choi, 1987).

Immunoliposomes loaded with either the dye or with calpain+caspase inhibitors alone or in combination are added to the media in the cell culture of CGN, which are then returned to incubation at 37° C. At 1 and 4 hour time intervals, the CGN cell culture plates are removed from the incubator, and fluorescent microscopy is performed after washing the cells to remove free floating liposomes.

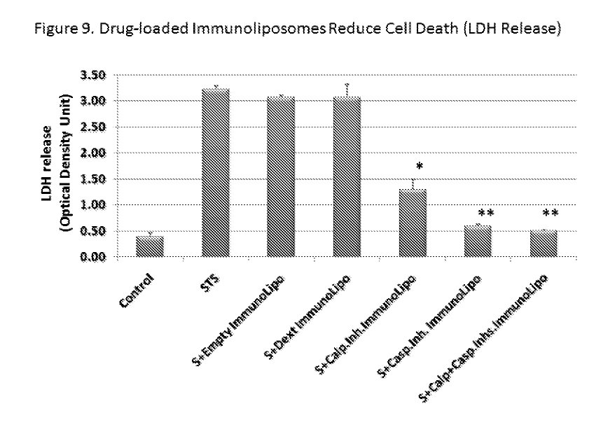

When compared with control cells, STS treated cells, as well as the negative controls using empty liposomes and Dextran-loaded liposomes show significant and comparable levels of cell death indicated by high concentrations of LDH in the cell media. (FIG. 9) Cells exposed to the calpain inhibitor, caspase inhibitor and the combined calpain and caspase inhibitor-loaded immunoliposomes demonstrate significant neuroprotection against STS-mediated cell death. (FIG. 9) (Statistical significance: *p<0.05, **p<0.01) Collectively, these data demonstrate that the NMDA-R-targeting immunoliposome nanosystem is effective in delivering neuroprotective drugs inside differentiated PC-12 cells to allow them to exert neuroprotective functions.

### Example 11

Administration of immunoliposomes decreases apoptotic markers in subject brain in vivo.

Immunoliposomes formulated with the calpain inhibitor (SJA6017) and caspase inhibitor (Z-D-DCB) as in Example 8 in immunoliposomes prepared as in Examples 4 or 5 are administered to subject cell cultures prior to (10 minutes to 6 hours) or following (immediate to 4 hours) brain injury by either TBI or MCAO.

In vivo model of TBI injury model: A controlled cortical impact (CCI) device is used to model TBI on rats as previously described (Pike et al, 1998). Adult male (280-300 g) Sprague-Dawley rats (Harlan: Indianapolis, Ind.) are anesthetized with 4% isoflurane in a carrier gas of 1:1 O₂/N₂O (4 min.) and maintained in 2.5% isoflurane in the same carrier gas. Core body temperature is monitored continuously by a rectal thermistor probe and maintained at 37±1° C. by placing an adjustable temperature controlled heating pad beneath the rats. Animals are mounted in a stereotactic frame in a prone position and secured by ear and incisor bars. Following a midline cranial incision and reflection of the soft tissues, a unilateral (ipsilateral to site of impact) craniotomy (7 mm diameter) is performed adjacent to the central suture, midway between bregma and lambda. The dura mater is kept intact over the cortex. Brain trauma is produced by impacting the right (ipsilateral) cortex with a 5 mm diameter aluminum impactor tip (housed in a pneumatic cylinder) at a velocity of 3.5 m/s with a 1.6 mm compression and 150 ms dwell time. Sham-injured control animals are subjected to identical surgical procedures but do not receive the impact injury. Appropriate pre- and post-injury management is preformed to insure compliance with guidelines set forth by the University of Florida Institutional Animal Care and Use Committee and the National Institutes of Health guidelines detailed in the Guide for the Care and Use of Laboratory Animals. In addition, research is conducted in compliance with the Animal Welfare Act and other federal statutes and regulations relating to animals and experiments involving animals and adhered to principles stated in the “Guide for the Care and Use of Laboratory Animals, NRC Publication, 1996 edition.”

Middle cerebral artery occlusion (MCAO) injury model: Rats are incubated under isoflurane anesthesia (5% isoflurane via induction chamber followed by 2% isoflurane via nose cone), the right common carotid artery (CCA) of the rat is exposed at the external and internal carotid artery (ECA and ICA) bifurcation level with a midline neck incision. The ICA is followed rostrally to the pterygopalatine branch and the ECA is ligated and cut at its lingual and maxillary branches. A 3-0 nylon suture is then introduced into the ICA via an incision on the ECA stump (the suture's path was visually monitored through the vessel wall) and advanced through the carotid canal approximately 20 mm from the carotid bifurcation until it becomes lodged in the narrowing of the anterior cerebral artery blocking the origin of the middle cerebral artery. The skin incision is then closed and the endovascular suture left in place for 30 minutes or 2 hours. Afterwards the rat is briefly reanesthetized and the suture filament is retracted to allow reperfusion. For sham MCAO surgeries, the same procedure is followed, but the filament is advanced only 10 mm beyond the internal-external carotid bifurcation and is left in place until the rat is sacrificed. During all surgical procedures, animals are maintained at 37±1° C. by a homeothermic heating blanket (Harvard Apparatus, Holliston, Mass., U.S.A.). It is important to note that at the conclusion of each experiment, if the rat brains show pathologic evidence of subarachnoid hemorrhage upon necropsy they are excluded from the study. Appropriate pre- and post-injury management is preformed to insure compliance with all animal care and use guidelines.

Following injury brain tissue from the area of damage is prepared. At the appropriate time points (2, 6, 24 hours and 2, 3, 5 days) after injury, animals are anesthetized and immediately sacrificed by decapitation. Brains are quickly removed, rinsed with ice cold PBS and halved. The right hemisphere (cerebrocortex around the impact area and hippocampus) is rapidly dissected, rinsed in ice cold PBS, snap-frozen in liquid nitrogen, and stored at −80° C. until used. For immunohistochemistry, brains are quick frozen in dry ice slurry, sectioned via cryostat (20 μm) onto SUPERFROST PLUS GOLD® (Fisher Scientific) slides, and then stored at −80° C. until used. For the left hemisphere, the same tissue as the right side is collected. For Western blot analysis, the brain samples are pulverized with a small mortar and pestle set over dry ice to a fine powder. The pulverized brain tissue powder is then lysed for 90 min at 4° C. in a buffer of 50 mM Tris (pH 7.4), 5 mM EDTA, 1% (v/v) Triton X-100, 1 mM DTT, 1× protease inhibitor cocktail (Roche Biochemicals). The brain lysates are then centrifuged at 15,000×g for 5 min at 4° C. to clear and remove insoluble debris, snap-frozen, and stored at −80° C. until used.

For gel electrophoresis and electroblotting, neuronal tissue is prepared as described in Example 8. cleared lysed brain tissue samples (7 μl) are prepared for sodium dodecyl sulfate-polyacrylamide gel electrophoresis (SDS-PAGE) with a 2× loading buffer containing 0.25 M Tris (pH 6.8), 0.2 M DTT, 8% SDS, 0.02% bromophenol blue, and 20% glycerol in distilled H₂O. Twenty micrograms (20 μg) of protein per lane are routinely resolved by SDS-PAGE on 10-20% Tris/glycine gels (Invitrogen, Cat #EC61352) at 130 V for 2 hours. Following electrophoresis, separated proteins are laterally transferred to polyvinylidene fluoride (PVDF) membranes in a transfer buffer containing 39 mM glycine, 48 mM Tris-HCl (pH 8.3), and 5% methanol at a constant voltage of 20 V for 2 hours at ambient temperature in a semi-dry transfer unit (Bio-Rad). After electro-transfer, the membranes are blocked for 1 hour at ambient temperature in 5% non-fat milk in TBS and 0.05% Tween-2 (TBST) then are incubated with the primary SBDP antibody in TBST with 5% non-fat milk at 1:2000 dilution as recommended by the manufacturer at 4° C. overnight. This is followed by three washes with TBST, a 2 hour incubation at ambient temperature with a biotinylated linked secondary antibody (Amersham, Cat #RPN1177v1), and a 30 min incubation with Streptavidin-conjugated alkaline phosphatase (BCIP/NBT reagent: KPL, Cat #50-81-08). Molecular weights of intact SBDP proteins are assessed using rainbow colored molecular weight standards (Amersham, Cat #RPN800V). Semi-quantitative evaluation of intact αII-Spectrin and SBDP protein levels is performed via computer-assisted densitometric scanning (Epson XL3500 scanner) and image analysis with ImageJ software (NIH).

Levels of SBDP145 in brain lysate are significantly (p<0.05) increased at all time points studied following severe (2 hr) MCAO challenge relative to mild (30 min) challenge in control studies. Similarly, SBDP120 demonstrates significant elevations following severe MCAO challenge between 24 and 72 hours after injury in CSF. The presence of inventive immunoliposomes containing calpain and caspase inhibitors decreases the levels of SBDP in neurons damaged by TBI or MCAO demonstrating neuroprotective or therapeutic effects in vivo.

The following references are each incorporated herein by reference as if the contents of each reference were fully and explicitly included.

### REFERENCES

Banerji, B. Kenny, J. J., Scher, I., Alving, C. R. (1982) Antibodies Against Liposomes in Normal and Immune-Defective Mice. The Journal of Immunology, 128(4) 1603-1607.

Charrier C, Ehrensperger M V, Dahan M, Levi S, Triller A. (2006) Cytoskeleton regulation of glycine receptor number at synapses and diffusion in the plasma membrane. The Journal of Neuroscience, 8502-8511.

Chiu, S J, Liu S, Perrotti D, Marcucci G, Lee R J. (2006) Efficient Delivery of a Bcl-2-Specific Antisense Oligodeoxyribonucleotide (G3139) Via Transferring Receptor-Targeted Liposomes. J Control Release. 112(2):199-207.

Hilgenbrink. A. R. Low, P. S. (2005) Folate Receptor-Mediated Drug Targeting: From Therapeutics to Diagnostics. J Pharm. Science 94(10) 2135-2146.

Hu, H., Chen, D, Liu, Y, Deng, Y, Yang, S, Qiao, M, Zhao, J, Zhao, X. (2006) Preparation and Targeted Delivery of Immunoliposomes Bearing Poly(Ethylene Glycol)-Coupled Humanized Anti-Hepatoma Disulfide-Stabilized Fv (HdsFv25) in Vitro. Pharmazie, 61(8) 685-688.

Jain. K. K. (2005) Nanotechnology-Based Drug Delivery for Cancer, Technol Cancer Res Treat: 4(4) 407-416.

Jones, R. (2006) What Can Biology Teach Us? Nature Nanotechnology 1, 85-86.

Langham, J. (2000) “Calcium Channel Blockers for Acute Traumatic Brain Injury.” The Cochrane Library. 2000;(2):CD000565. The Cochrane Collaboration. Retrieved on 2, Nov. 2007 from <http://www.cochrane.org/reviews/en/ab000565.html>.

Liang, W. (2004) ATP-Containing Immunoliposomes Specific for Cardiac Myosin. Current Drug Delivery, 1: 1-7.

Nasongkla, N., Bey, E., Ren, J., Ai, H., Khemtong, C., Guthi, J. S., Chin, S. F., Sherry, A. D., Boothman, D. A., Gao, J. (2006) Multifunctional Polymeric Micelles as Cancer-Targeted, MRI-Ultrasensitive Drug Delivery Systems. Nano Lett. 6(11):2427-2430.

Institute for the Translational Medicine and Therapeutics (2005) “Program in Targeted Therapeutics (PTT).” Penn Medicine. Retrieved on 27 Nov. 2007 from http://www.itmat.upenn.edu/research_ptt.shtml

Racca, C. Gardiol A, Triller A. (1997) Dendritic and Postsynaptic Localizations of Glycine Receptor alpha subunit mRNAs. The Journal of Neuroscience, 17(5) 1691-1700.

Rosenberg, M., Meier J, Triller A, Vannier C. (2001) Dynamics of Glycine Receptor Insertion in the Neuronal Plasma. The Journal of Neuroscience, 21 (14) 5036-5044.

Sachdeva, M. S. (1998) Drug Targeting Systems for Cancer Chemotherapy. Expert Opinion Investigating Drugs, 7(11); 1849-1864.

Sharma G, Anabousi S, Ehrhardt C, Ravi Kumar M N. (2006) Liposomes as Targeted Drug Delivery Systems in the Treatment of Breast Cancer. J Drug Target 14(5): 301-310.

Torchilin, V. P. (2005) Fluorescence Microscopy to Follow the Targeting of Liposomes and Micelles to Cells and Their Intracellular Fate. Adv Drug Deliv Rev.; 57(1):95-109.

Nichcy. (2006) “Traumatic Brain Injury.” Disability Info. May 2007. Retrieved on 27 Nov. 2006 from <http://www.nichcy.org/pubs/factshe/fs18txt.htm#idea>.

Umamaheshwari, R. B. Suman Ramteke, 1 and Narendra Kumar (2004) Jain Anti-Helicobacter Pylori Effect of Mucoadhesive Nanoparticles Bearing Amoxicillin in Experimental Gerbils Model. AAPS PharmSciTech. April 7; 5(2):e32.

Sadasivan, S., Waghray, A., Lamer, S. F., William A. Dunn, W. A. Jr., Hayes, R. L. and Wang, K. K. W. (2006). Amino Acid Starvation Induced Autophagic Cell Death in PC-12 Cells: Evidence for Activation of Caspase-3 but not Calpains. Apoptosis 11(9):1573-1582

Singh, Hari Gour University. PharmSci Tech, 2004. Retrieved on 27 Nov. 2007 from <http://www.aapspharmscitech.org/view.asp?art=pt050232&pdf=yes>.

Wang, K. K. W. and Yuen, P. w. (1994) Calpain inhibition: an overview of its therapeutic potentials. Trends Pharmacol. Sci. 15, 412-419.

Wang, K. K. W. (2000) Calpain and Caspase: Can You Tell the Difference. Trends Neurosci. 23, 20-26.

Yih. T. C. and Al-Fandi, M. (2006) Engineered Nanoparticles as Precise Drug Delivery Systems, by. J Cell Biochem. 97(6): 1184-1190.

Zangemeister-Wittke, U. and Z Zangemeister-Wittke, U. (2005) Antibody for Targeted Cancer Therapy—Pathobiology 72: 279-286.

Patents and publications mentioned in the specification are indicative of the levels of those skilled in the art to which the invention pertains. These patents and publications are incorporated herein by reference to the same extent as if each individual application or publication was specifically and individually expressed explicitly in detail herein.

The foregoing description is illustrative of particular embodiments of the invention, but is not meant to be a limitation upon the practice thereof. The following claims, including all equivalents thereof, are intended to define the scope of the invention.

1. A process for delivering an active agent molecular cargo into a neuronal cell comprising:
placing at least one active agent cargo molecule within a synthetic vesicle;
binding a biotinylated protein to said synthetic vesicle to form a protein bound synthetic vesicle, said protein recognizing a receptor expressed on the surface of a neuronal cell; and
exposing said cell to said protein bound synthetic vesicle until said at leatone active agent cargo molecule is delivered into the neuronal cell.

2. The process of claim 1 wherein said protein is an antibody.

3. The process of claim 1 wherein said at least one active agent cargo molecule is a calpain inhibitor, a caspase inhibitor, or combination thereof.

4. A process of claim 1 wherein said synthetic vesicle is a liposome.

5. The process of claim 1 wherein said receptor is a cellular receptor for a ligand selected from the group comprising: glutamate, glycine, dopamine, nicotine, muscarine, acetylcholine, or serotonin.

6. The process of claim 1 wherein said at least one active agent cargo molecule is loaded into said synthetic vesicle before binding a biotinylated protein thereto.

7. The process of claim 1 wherein said synthetic vesicle is bound to said biotinylated protein with avidin or streptavidin intermediate there between.

8. The process of claim 1 wherein said neuronal cell is in vitro or ex vivo.

9. The process of claim 1 wherein said neuronal cell is in vivo.

10. The process of claim 9 wherein said neuronal cell is in the central nervous system of a subject.

11. The process of claim 9 wherein said neuronal cell is in the peripheral nervous system of a subject.

12. A composition comprising:
a synthetic vesicle having a volume and an exterior surface;
at least one active agent cargo molecule within the volume of said synthetic vesicle
a biotinylated antibody bound to the exterior surface of said synthetic vesicle, said antibody recognizing a receptor expressed on the surface of a neuronal cell.

13. The composition of claim 12 wherein said receptor is a cellular receptor for a ligand selected from the group comprising: glutamate, glycine, dopamine, nicotine, muscarine, acetylcholine, or serotonin.

14. The composition of claim 12 wherein said ligand is glutamate.

15. The composition of claim 12 wherein said synthetic vesicle is a liposome, poly(lactic-co-glycolic acid) or polycaprolactone.

16. The composition of claim 12 further comprising avidin or streptavidin intermediate between said biotinylated antibody and said synthetic vesicle.

17. The composition of claim 12 wherein said synthetic vesicle is biotinylated.

18. The composition of claim 12 wherein said at least one active agent cargo molecule is a calpain inhibitor, a caspase inhibitor, or combinations thereof.

19. A method of treating a disease, injury or condition of a CNS cell comprising administering the composition of claim 12 to a subject.

20. The method of claim 19 wherein said condition is traumatic brain injury.

21. The method of claim 19 wherein said at least one active agent cargo molecule is an apoptosis or oncosis inhibitor.

22. The method of claim 19 wherein said apoptosis or oncosis inhibitor is selected from the group comprising a calpain inhibitor, a caspase inhibitor, or combinations thereof.

23. The method of claim 22 wherein said treating is by delivering said apoptosis or oncosis inhibitor to a CNS cell exposed to trauma.

24. A process of making the compound of claim 12 comprising:
forming a synthetic vesicle; and
incorporating biotinylated phosphatidylethanolamine into said outer surface exclusively.

25. The process of claim 24 wherein said at least one active agent cargo molecule is present while forming said synthetic vesicle.

26. The process of claim 24 wherein avidin or streptavidin is bound to said antibody prior to said antibody binding to said synthetic vesicle.

27. A formulation which comprises as an active ingredient a composition of claim 12 associated with one or more acceptable carriers, excipients or vehicles therefor

28. (canceled)

29. A commercial package comprising a composition of claim 12 as an active ingredient together with instructions for the use thereof for delivery of an active agent cargo molecule to a neuronal cell.

30. (canceled)
